(19) United States
(12) Patent Application Publication
(10) Pub. No.: US 2010/0256436 A1
(43) Pub. Date: Oct. 7, 2010

(54) DEVICE AND METHOD FOR TREATING HYPERTENSION VIA NON-INVASIVE NEUROMODULATION

(76) Inventors: Michael J. Partsch, Redwood City, CA (US); M. Bret Schneider, Portola Valley, CA (US); David J. Mishelevich, Playa del Rey, CA (US)

Correspondence Address: SHAY GLENN LLP, 2755 CAMPUS DRIVE, SUITE 210, SAN MATEO, CA, 94403, US

(21) Appl. No.: 12/669,882

(22) PCT Filed: Jul. 30, 2008
(86) PCT No.: PCT/US08/71663

§ 371 Date: Jun. 2, 2010

Related U.S. Application Data
(60) Provisional application No. 60/953,191, filed on Jul. 31, 2007.

(51) Int. Cl. A61N 2/00

(52) U.S. Cl. 600/9

(57) ABSTRACT

Hypertension may be caused by central nervous system-mediated effort to maintain a certain level of blood flow within the brain. A method is described for using neuromodulation techniques to lower central drive for hypertension.

### CROSS REFERENCE TO RELATED APPLICATIONS

This application claims priority to U.S. Provisional Patent Application Ser. No. 60/953,191, filed on Jul. 31, 2007, titled “DEVICE AND METHOD FOR TREATING HYPERTENSION VIA NON-INVASIVE NEUROMODULATION.”

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The devices and methods described herein relate generally to the treatment of hypertension.

### BACKGROUND OF THE INVENTION

Arterial hypertension, commonly referred to as “hypertension” or “high blood pressure”, is a medical condition in which the blood pressure is chronically elevated. Hypertension is associated with markedly elevated risk of heart attack, heart failure, arterial aneurysms, kidney failure and stroke. Causes of hypertension in a given individual may be one or more of many possibilities, which may include salt intake, obesity, occupation, alcohol intake, smoking, family size, stimulant intake, excessive noise exposure, and crowding, renin levels, insulin resistance, sleep apnea, genetic susceptibility, decreased kidney perfusion, catecholamine-secreting tumors of the adrenal glands, Adrenal hypertension with aldosterone-induced sodium retention, hypercalcemia, coarctation of the aorta, diet, medications, arterial stiffening that accompanies age. When the hypertension is secondary to another medical condition, it is generally prudent to treat that primary condition first. However, regardless as to whether the hypertension is primary or secondary, the blood pressure typically is subject to modification by several different approaches including changing (typically via medications) fluid excretion, heart activity, and blood vessel contraction.

Medications for blood pressure control are frequently not effective, or present troublesome side effects when raised to a therapeutic dose. Depending on the class of medication, such side effects range from the inconvenient to the deadly, and may include constipation, edema, exercise intolerance, impotence, orthostasis, syncope and stroke.

One frequently overlooked avenue for modifying blood pressure is control of the pressure by mechanisms intrinsic to the brain. The brain is a highly metabolically active organ with an immense need for oxygen-rich blood. When mechanisms within the brain sense low blood flow, mechanisms including those within the brain stem activate to raise overall blood pressure to levels adequate for perfusion of the brain, so as to avoid hypoxia. This may lead to a systemic hypertension.

Baroreceptors in the human body detect the pressure of blood flowing through them, and send messages to the central nervous system to increase or decrease total peripheral resistance and cardiac output, and thereby change blood pressure. There are baroreceptors in locations including the arch of the aorta, and the carotid sinuses of the left and right internal carotid arteries. Baroreceptors act to maintain mean arterial blood pressure to allow tissues to receive the right amount of blood. Neural signals from the baroreceptors are processed within the brain, in order to maintain physiological homeostasis. For example, the solitary nucleus and tract within the medulla and pons, receive signals from the carotid and aortic baroreceptors. In response to a perception of low blood pressure, the solitary nucleus sends out signals leading to hypertension, tachycardia and sympatho-excitation. In response to a perceived state of high blood pressure, the opposite physiological response is triggered.

There are known methods for selectively increasing blood flow within the brain. One such method is transcranial magnetic stimulation (Speer et al 2000, Conca et al 2002, Takano et al 2004, Ohnishi et al 2004). The increased blood flow appears to occur as a result of the increased metabolic demands of activated neurons. Increased blood flow effect appears to be sustained long after the rTMS stimulus was received: In the case of rTMS-treated depression for example, the clinical benefit is closely associated with the enhance blood flow in the left dorsolateral prefrontal cortex, and appears to last for months before resuming its former pattern.

While it has been demonstrated that stimulation of the solitary nucleus with an implanted electrode serves to lower blood pressure, this method requires invasive brain surgery, the risks of which outweigh the benefits of treating hypertension in such a manner. The prior art does not show means by which blood pressure is lowered by non-invasive brain stimulation.

### SUMMARY OF THE INVENTION

Described herein are methods and devices for treating hypertension by non-invasive techniques. In particular, described herein are devices and methods for treating hypertension by transcranial magnetic stimulation of one or more regions of a subject's brain. For example, described herein are methods for selectively modulating neuronal tissue so as to prompt central nervous system mechanisms to lower systemic blood pressure. These methods may include the steps of magnetically stimulating a brain region (e.g., using transcranial magnetic stimulation) to controllably trigger central homeostatic mechanisms, resulting the lowering of the subject's blood pressure drive. In direct modulation of blood pressure using central homeostatic mechanisms, brain regions that may be targeted for stimulation include the superficial cortical aspects of the frontal lobes, the parietal lobes, the temporal lobes, the occipital lobes, or the cerebellum. For direct modulation of blood pressure, appropriate brain regions that may be stimulated include: solitary nucleus and tract or related brainstem circuitry.

As mentioned, stimulation may be transcranial stimulation. For example, transcranial stimulation may be electromagnetic pulses of approximately 1 Tesla in intensity, each lasting approximately 100 microseconds. Application of these pulses at frequencies of approximately 1 Hz to 25 Hz, for approximately 45 minutes, and repeated for several consecutive days generally serve to change activity level in the targeted brain region for 2-7 months.

In one embodiment, herein termed the “indirect” method, brain tissue is stimulated so as to increase its metabolic rate, leading to increased blood flow in the stimulated tissue. Secondarily, central homeostatic mechanisms lower their blood pressure drive. In some variations, the subject may be monitored, and the stimulation linked to feedback from the subject.

In addition to stimulation by transcranial magnetic stimulation, other types of stimulation may be used These may include ultrasound, pulsed electrical currents and direct electrical currents.

In an alternative embodiment, brainstem blood pressure homeostatic mechanisms are stimulated “directly” using transcranial brain stimulation. By this approach, the solitary nucleus or related circuitry within the brainstem is stimulated so as to directly inhibit blood pressure through intrinsic, dedicated physiological methods.

Pulse generation devices that produce such pulses are commercially available such as the Magstim Rapid stimulator by Magstim LTD (Wales, UK). When used with commercially available coils such as the 70 mm double coil (Magstim LTD (Wales, UK)), such stimulators may be used a rates of 5 Hz or greater to increase blood flow in the cortical sufaces of the frontal, temporal, parietal and occipital lobes as well as in the cerebellum. This increase in blood flow may be used to increase blood flow to the targeted structures, thereby invoking the “indirect” method as herein described. When an array of stimulators, configured to power multiple coils simultaneously as herein described, stimulation may be targeted toward deeper, subcortical brain structures including the solitary nucleus and tract of the brainstem.

### BRIEF DESCRIPTION OF THE DRAWINGS

FIG. 1 outlines the basic steps of the method described herein.

FIG. 2 illustrates examples of several exemplary anatomic locations for interventions in accordance with the methods herein described.

FIGS. 3A and 3B illustrate the use of a multiple-coil array in order to stimulate the brainstem, in particular the solitary tract and nucleus.

### DETAILED DESCRIPTION OF THE INVENTION

In general, methods of lower systemic blood pressure in accordance with the present invention include

1) an “indirect” method in which overall cerebral blood flow is induced to rise using transcranial magnetic stimulation or other transcranial approach to the frontal, temporal, parietal, or occipital cortex, or to the cerebellum, thereby secondarily triggering to lowering of blood pressure regions, and

2) a “direct” method in which the solitary nucleus and tract of the brainstem or related circuitry is directly stimulated using transcranial magnetic stimulation or other transcranial approach.

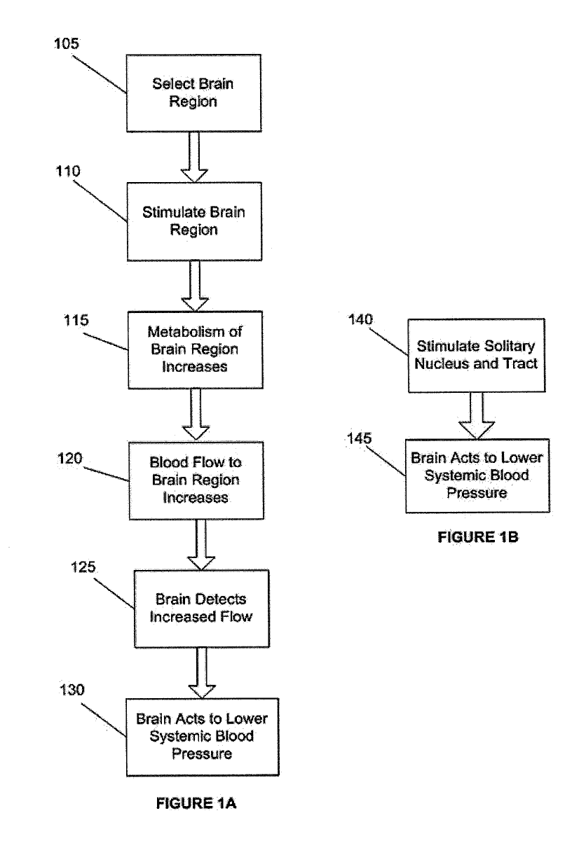

FIGS. 1A, 1B, and 1C outline the basic steps of some of the methods described herein.

In FIG. 1A, a method is described in which increasing cerebral perfusion in general is used to effect an antihypertensive treatment. In step 105 a region of a hypertensive patient is studied, and a region of that patient's brain in selected to be the primary target for stimulation. This region could be the entire brain, for example using a large coil or a coil array that encircles the head, or could be a specific region, for example the brainstem. In step 110 that region is stimulated in a manner that is anticipated to increase blood flow in the brain or brainstem. Stimulation means may include repetitive transcranial magnetic stimulation (rTMS), stereotactic transcranial magnetic stimulation (sTMS) as described in U.S. Ser. No. 10/821,807, U.S. Ser. No. 11/429,504. Stimulation means may also include the implanted electrodes of deep brain stimulation (DBS) as is known in the art, superficial cortical stimulation grids, and transcranial direct current stimulation (tDCS) (Lang, et al 2005). Steps 115 through 130 describe the physiological responses that are evoked in response to targeted stimulations cited stimuli act upon those physiological circuits as described above. In step 115, the body is stimulated to increase the metabolism of the stimulated brain region. For example, in step 120, the body is stimulated to increase blood flow to the stimulated area. In step 125, stimulation proceeds until the brain detects this increased flow with its intrinsic pressure receptor and flow sensors. Areas in which these sensors are located are probably many, and include the solitary tract and nucleus (nucleus tractus solitarii) (Paton et al 2007, Waki et al 2007, Vayssettes-Courchay et al 1993). In step 130, the stimulation leads to detection of increased blood flow, triggering lower blood pressure through its intrinsic mechanisms, including downregulation of vascular tone, cardiac output, and blood volume.

FIG. 1B outlines a method by which hypertension is treated by direct stimulation of the solitary nucleus and tract. In step 140, the solitary nucleus and tract are stimulated, for example magnetically using a transcranial magnetic stimulation as shown in the subsequent figures and description, for example at a pulse rate of 5 Hz, 3500 pulses delivered per day for 20 consecutive weekdays. In step 145, the nucleus interprets this stimulation as signals of hypertension, in much the same manner that it normally interprets such activity as input from the baroreceptors in the carotid bodies and aortic bodies, and acts to lower blood pressure by its endogenous abilities based on the stimulation.

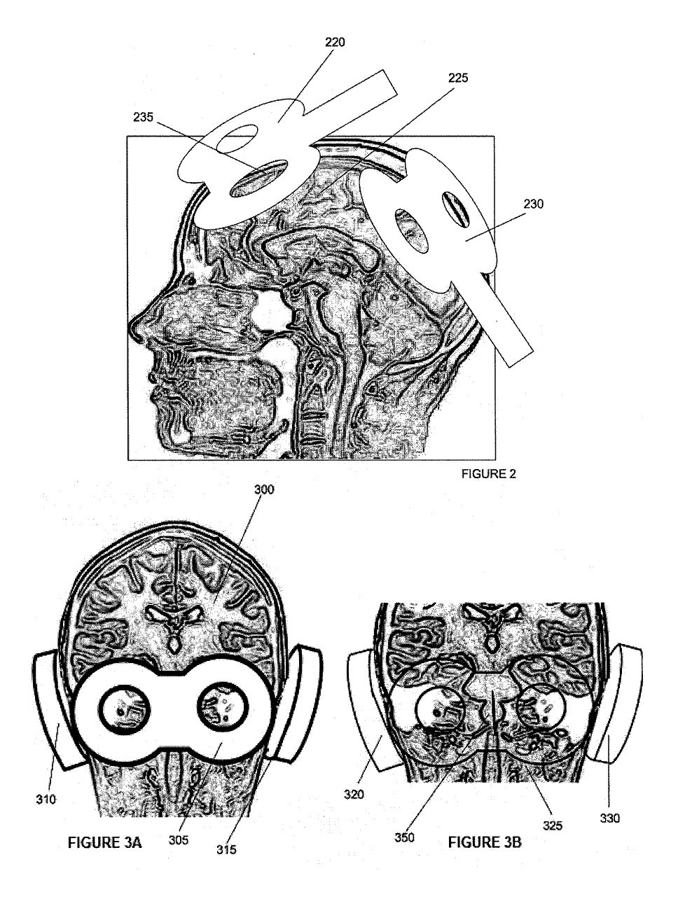

FIG. 2 illustrates examples of two anatomic locations for interventions in accordance with the steps outlined in FIG. 1A. TMS coil 220 and TMS coil 230 may be 70 mm air-cooled double coil attached to a Rapid2 stimulator machine (Magstim Ltd., Wales, UK), and are shown in perspective to represent placement at an angle to the viewing plane. TMS coil 220 is centered over brain area target 235. TMS coil 230 is shown placed in a posterior parietal location, as an example of an alternate placement.

FIGS. 3A and 3B shows the use of multiple-coil arrays, like those described in “Robotic device for stereotactic transcranial magnetic stimulation.” Schneider MB and Mishelevich DJ U.S. Ser. No. 10/821,807, and in “Trajectory-Based Transcranial Magnetic Stimulation” Mishelevich DJ and Schneider MB, Pending U.S. patent application Ser. No. 11/429,504. In FIG. 3a, Coils 305, 310, and 315 surround the posterior aspect of the head, and may be moveable or stationary. In FIG. 3a the central coil 325 (equivalent to coil 305 in FIG. 3a) is shown in transparency. Solitary nucleus and tract 350 is shown in the lower pons and the medulla, roughly beneath the center of coil 325. Coil 320 and 330 are equivalent to coil 210 and 315, respectively.

### REFERENCES

Ohnishi T, Matsuda H, Imabayashi E, Okabe S, Takano H, Arai N, Ugawa Y. rCBF changes elicited by rTMS over DLPFC in humans. Suppl Clin Neurophysiol. 2004;57:715-20.

Takano B, Drzezga A, Peller M, Sax I, Schwaiger M, Lee L, Siebner H R. Short-term modulation of regional excitability and blood flow in human motor cortex following rapid-rate transcranial magnetic stimulation. Neuroimage. 2004 November;23(3):849-59.

Conca A, Peschina W, Konig P, Fritzsche H, Hausmann A. Effect of chronic repetitive transcranial magnetic stimulation on regional cerebral blood flow and regional cerebral glucose uptake in drug treatment-resistant depressives. A brief report. Neuropsychobiology. 2002;45(1):27-31.

Speer A M, Kimbrell T A, Wassermann E M, D Repella J, Willis M W, Herscovitch P, Post R M. Opposite effects of high and low frequency rTMS on regional brain activity in depressed patients. Biol Psychiatry. 2000 Dec. 15;48(12):1133-41.

Paton J F, Waki H, Abdala A P, Dickinson J, Kasparov S. Vascular-brain signaling in hypertension: role of angiotensin II and nitric oxide. Curr Hypertens Rep. 2007 June;9(3):242-7.

Vayssettes-Courchay C, Bouysset F, Verbeuren T J, Laubie M. Role of the nucleus tractus solitarii and the rostral depressive area in the sympatholytic effect of 8-hydroxy-2-(di-n-propylamino) tetralin in the cat. Eur J Pharmacol. 1993 Sep. 21;242(1):37-45.

Waki H, Liu B, Miyake M, Katahira K, Murphy D, Kasparov S, Paton J F. Junctional adhesion molecule-1 is upregulated in spontaneously hypertensive rats: evidence for a prohypertensive role within the brain stem. Hypertension. 2007 June;49(6):1321-7.

Lang N, Siebner H R, Ward N S, Lee L, Nitsche M A, Paulus W, Rothwell J C, Lemon R N, Frackowiak R S. How does transcranial DC stimulation of the primary motor cortex alter regional neuronal activity in the human brain? Eur J Neurosci. 2005 July;22(2):495-504.

“Robotic device for stereotactic transcranial magnetic stimulation.” Schneider M B and Mishelevich D J U.S. Ser. No. 10/821,807

“Trajectory-Based Transcranial Magnetic Stimulation” Mishelevich DJ and Schneider M B, Pending U.S. patent application Ser. No. 11/429,504

What is claimed is:

1. A method for lowering human blood pressure comprising:
directing energy toward the neural tissue so as to increase brain metabolism and blood flow, thereby lowering central blood pressure drive.

2. A method as in claim 1 wherein said energy source is transcranial magnetic stimulation.

3. A method as in claim 1 in which the solitary nucleus and tract signal to decrease the blood flow.

4. A method for treating hypertension comprising:
stimulating brain tissue so as to increase blood flow within the brain, thereby raising central nervous system drive to decrease blood pressure.

5. A method for treating hypertension comprising:
stimulating brain tissue so as to increase blood flow within the brain, thereby lowering central nervous system drive to increase blood pressure.

6. A method for treating hypertension comprising:
stimulating brain tissue so as to increase blood flow within the brain, thereby raising central nervous system drive to decrease blood pressure.
